(19) United States
(12) Patent Application Publication
(10) Pub. No.: US 2025/0268508 A1
(43) Pub. Date: Aug. 28, 2025

(54) INTER-BEAT INTERVAL SEQUENCE OF HEART FOR ESTIMATING CONDITION OF SUBJECT

(71) Applicant: Tampere University Foundation sr, Tampere (FI)

(72) Inventors: Esa RÄSÄNEN, Tampere (FI); Matti MOLKKARI, Tampere (FI)

(21) Appl. No.: 19/180,553

(22) Filed: Apr. 16, 2025

Related U.S. Application Data
(63) Continuation of application No. PCT/FI2023/050738, filed on Dec. 22, 2023.

(30) Foreign Application Priority Data
Dec. 22, 2022 (FI) 20226156

(51) Int. Cl. A61B 5/352; A61B 5/00

(52) U.S. Cl. CPC A61B 5/352; A61B 5/40; A61B 5/4812; A61B 5/4842

(57) ABSTRACT

There is provided a method for estimating a condition of a subject based on a simple heart rate measurement. The method comprises obtaining an inter-beat interval (IBI) sequence of a heart; detrending fluctuations derivable from the obtained IBI sequence at a plurality of window lengths; determining variances of the detrended fluctuations for the plurality of window lengths; determining first distributions of powers of the plurality of window lengths based on proportionality of the powers of the window lengths to root mean values of the determined variances, wherein the first distributions are determined over at least one quantity characterizing the obtained IBI sequence; determining second distributions of powers for the plurality of window lengths over the at least one quantity characterizing the obtained IBI sequence; and estimating a condition of a subject, or a change of a condition of a subject, based on comparing the determined first distributions with the determined second distributions.

### CROSS-REFERENCE TO RELATED APPLICATIONS

This application is a continuation of International Application PCT/FI2023/050738, filed Dec. 22, 2023, which claims priority to and the benefit of Finnish Application No. 20226156, filed on Dec. 22, 2022, the contents of which are expressly incorporated by reference herein in their entireties.

### TECHNICAL FIELD

Various example embodiments relate to an inter-beat interval sequence of heart and more particularly using the inter-beat interval sequence of a heart for estimating condition of a subject.

### BACKGROUND

Cardiac diagnosis and health assessment contains a multitude of methods and techniques, e.g., electrocardiogram (ECG) analysis, treadmill stress tests, echocardiography (ECHO), single photon emission computed tomography (SPECT), magnetic resonance imaging (CMR) and positron emission tomography (PET). All of these methods require the use of advanced and expensive technology; minimally a multichannel ECG device such as a Holter monitor.

Outside the clinical context, simple heart rate (HR) monitors and fitness trackers that record only the HR intervals have become very common in the population to assess, e.g., the training intensity and recovery by monitoring the HR and/or the HR variability (HRV). This information is then conveyed to the consumer in a simple form including, e.g., the HR, the HR related to the maximum HR in terms of training intensity, or the HRV in terms of the level of recovery, or the state of the parasympathetic nervous system. HRV is typically computed using simple measures in the time- and frequency domain including, e.g., the standard deviation of the successive beat-to-beat intervals (SDNN), the root mean square of the intervals (RMSSD), or the ratio between low- and high-frequency components (LF/HF).

Therefore, clinical assessment of cardiac health, including also portable ECG devices for pre-diagnostics, and HRV tracking with consumer electronics, are still very far apart. On one hand, the above-mentioned hardware solutions for clinical assessment, including the required cardiological expertise and workforce, are very expensive and complicated. On the other hand, the above-mentioned HRV measures in wearable consumer electronics are not sufficiently precise and/or comprehensive for clinical assessment or for pre-diagnostics. Although HR and HRV can be used for detecting a likely atrial fibrillation, broader use of HR and HRV is limited due to differences between persons regarding the HR and HRV.

### SUMMARY

The problem mentioned above is alleviated by providing a method and technical equipment, where the method is implemented. Various aspects comprise a method, an apparatus, and a computer program product comprising a computer program stored therein, which are characterized by what is stated in the independent claims. Various example embodiments are disclosed in the dependent claims.

In a first aspect, the invention provides a method comprising
obtaining an inter-beat interval (IBI) sequence of a heart;
detrending fluctuations derivable from the obtained IBI sequence at a plurality of window lengths.
determining variances of the detrended fluctuations for the plurality of window lengths;
determining first distributions of powers of the plurality of window lengths based on proportionality of the powers of the window lengths to root mean values of the determined variances, wherein the first distributions are determined over at least one quantity characterizing the obtained IBI sequence;
determining second distributions of powers for the plurality of window lengths over the at least one quantity characterizing the obtained IBI sequence; and
estimating a condition of a subject, or a change of a condition of a subject, based on comparing the determined first distributions with the determined second distributions.

In a second aspect, the invention provides an apparatus comprising means for carrying out the method.

In a third aspect, the invention provides a computer program comprising computer program code configured to, when executed on at least one processor, cause an apparatus to perform the method.

The features recited in the dependent claims and the embodiments in the description are mutually freely combinable unless otherwise explicitly stated. The exemplary embodiments presented in this text and their advantages relate by applicable parts to all aspects of the invention, even though this is not always separately mentioned.

An advantage of the present invention is that cardiac health may be assessed using simple instrumentation and utilizing a simple heart rate measurement. The use of advanced and expensive technology may be avoided by using the present invention.

The present invention provides a novel method for processing heart rate data in the form of inter-beat interval (IBI) sequences. The IBI sequence data may be processed in a precise and comprehensive manner to provide a preliminary clinical assessment or a pre-diagnosis without the use of complex and expensive hospital-grade technology.

The present invention provides a way to assess the clinical condition of a subject with the use of a simple instrument utilizing the method provided herein.

### DESCRIPTION OF THE DRAWINGS

In the following, various example embodiments will be described in more detail with reference to the appended drawings, in which

FIG. 1 shows, by way of example, an ECG signal for forming inter-beat interval (IBI) sequence of a heart;

FIG. 2 shows, by way of example, a block diagram of an apparatus for estimating condition of subject;

FIG. 3 illustrates, by way of example, a method for estimating a condition of a subject;

FIG. 4 shows, by way of example, distributions that have been determined based on measured IBI sequences;

FIG. 5 shows examples of aggregated distributions for various conditions of subjects;

FIG. 6 shows examples of distributions for various conditions of a single subject;

FIG. 7 shows examples of distributions for determining exercise load of a subject; and

FIG. 8 shows examples of aggregated day and night distributions for healthy subjects and subjects with LQTS.

### DESCRIPTION OF EXAMPLE EMBODIMENTS

The following embodiments are exemplary. Although the specification may refer to “an”, “one”, or “some” embodiment(s) in several locations, this does not necessarily mean that each such reference is to the same embodiment(s), or that the feature only applies to a single embodiment. Single features of different embodiments may also be combined to provide other embodiments.

Use of ordinal terms such as “first,” “second,” “third,” etc., in the claims and description to modify a described feature does not by itself connote any priority, precedence, or order of one described feature over another or the temporal order in which acts of a method are performed, but are used merely as labels to distinguish one described feature having a certain name from another described feature having a same name (but for use of the ordinal term) to distinguish the described feature.

As used herein, “at least one of the following:” and “at least one of” and similar wording, where the list of two or more elements are joined by “and” or “or”, mean at least any one of the elements, or at least any two or more of the elements, or at least all the elements. There is provided method comprising obtaining an inter-beat interval (IBI) sequence of a heart; detrending fluctuations derivable from the obtained IBI sequence at a plurality of window lengths; determining variances of the detrended fluctuations for the plurality of window lengths; determining first distributions of powers of the plurality of window lengths based on proportionality of the powers of the window lengths to root mean values of the determined variances, wherein the first distributions are determined over at least one quantity characterizing the obtained IBI sequence; determining second distributions of powers for the plurality of window lengths over the at least one quantity characterizing the obtained IBI sequence; and estimating a condition of a subject, or a change of a condition of a subject, based on comparing the determined first distributions with the determined second distributions. The IBI sequence of the heart may be an IBI sequence of the heart of a subject. The method facilitates estimating condition of subject based on simple instrumentation and utilizing a simple heart rate measurement. Examples of the simple instrumentation comprise monitoring devices capable of measuring electrical activity of the heart and devices capable of measuring activity of the heart based on photoplethysmography. The monitoring devices may be wearable heart rate sensors, smart rings, sport watches or ECG monitoring devices. Result of the measurement of the electrical activity of the heart may be a signal for indicating an IBI sequence of the heart. Examples of the signal comprise an ECG signal and a beat rate signal. The beat rate signal may be a sample sequence comprising instantaneous values of heart rate, or beat rate, of the heart.

IBI sequence is a characteristic of a heart of a subject and comprises sample values that indicate time intervals between successive heart beats of the heart. The IBI sequence may be determined by simple instrumentation and utilizing a simple heart rate measurement. However, it should be noted that, the IBI sequence may be determined also by more sophisticated instrumentation such as an ECG device that is capable of measuring an ECG signal from a subject. The successive heart beats, or beats, may be determined based on an RR interval derived from the ECG signal.

FIG. 1 shows, by way of an example, an ECG signal for forming inter-beat interval (IBI) sequence of a heart. The ECG signal 100 may be measured from a subject and the measured ECG signal may be sampled for forming the IBI sequence. The ECG signal comprises heart beats, i, at RR intervals 101, 102, 104, 106, 108, 110, 112. The RR interval is a beat-to-beat interval which is calculated as the time between successive R-peaks. The RR intervals, therefore, form the IBI sequence. The RR intervals depend on various factors such as age, gender, and/or physiological status of a person. Physiological status here may refer to cardiac health, rest, sleep, exercise, anxiety, etc. Therefore, the IBI sequence can be used for obtaining information for estimating at least one of the following: cardiac health; or exercise load; or drug exposure; or sleep phase; or stress level; or and state of nervous system. The cardiac health may comprise at least one of the following: a healthy heart of the subject; or a cardiac disease of the subject; or a cardiac malfunction of the subject.

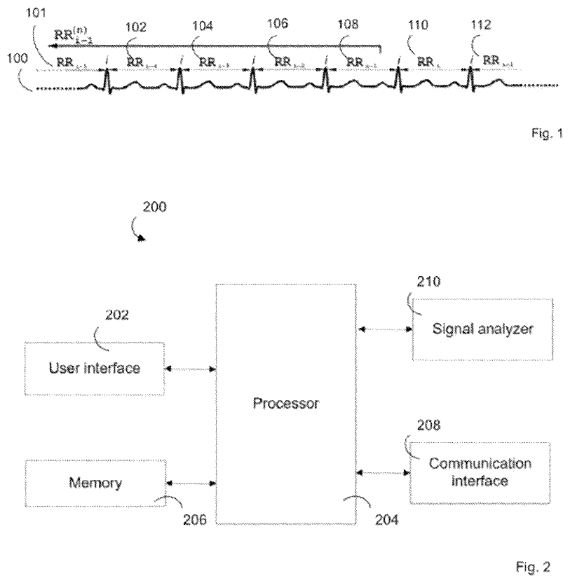

FIG. 2 shows, by way of an example, a block diagram of an apparatus 200 for estimating condition of subject. The apparatus may be e.g. a server or a computer or a smart phone. Alternatively, the apparatus may be or may comprise an ECG monitoring device, such as a Holter machine or a large-scale ECG monitor. Alternatively, the apparatus may be a wearable monitoring device, e.g. a sport watch, smart ring or any wearable heart rate monitor capable of measuring a signal representing electrical activity of the heart of a user of the wearable monitoring device and determining an IBI sequence from the signal. Examples of the signal comprise at least an ECG signal and a beat rate signal. The apparatus may receive user input such as commands, parameters etc. via a user interface 202 and/or via a communication interface 208. Examples of the commands comprise at least one of the following: a command to reset an alarm; or a command to estimate a condition of a subject; start or stop estimating a condition of a subject recording; or start or stop transfer of IBI sequence from the apparatus to a remote apparatus for estimating a condition of a subject by the remote apparatus. The user interface may receive user input e.g. through buttons and/or a touch screen. Alternatively, the user interface may receive user input from the Internet or a personal computer or a smartphone via a communication connection. The communication connection may be e.g. the Internet, a mobile communication network, Wireless Local Area Network (WLAN), Bluetooth®, or other contemporary and future networks. The apparatus may comprise a memory 206 for storing data and computer program code which may be executed by a processor 204 to carry out various embodiments of the method as disclosed herein. A signal analyzer 210 may be configured to implement the elements of the method disclosed herein. The signal analyzer may receive a signal to be processed, e.g. an ECG signal or a beat rate signal, from the memory or from a device, e.g. the heart rate monitoring unit or a wearable monitoring device, capable of measuring a signal representing electrical activity of the heart. The elements of the method may be implemented as a software component residing in the apparatus. The apparatus may receive the signal to be processed e.g. from a monitoring device and store the signal in the memory. The monitoring device may be any ECG hardware or a wearable monitoring device as described above. A computer program product may be embodied on a non-transitory computer readable medium. The apparatus may comprise means such as circuitry and electronics for handling, receiving and transmitting data, such as an ECG signal, a beat rate signal, an IBI sequence and/or a condition of a subject.

It should be appreciated that at least in some embodiments, the apparatus 200 may provide an output via the user interface and/or the communication interface 208. The output may comprise output data that is displayed by the user interface or output data that is communicated by the communication interface 208. In an example the output may comprise displaying of information, by the user interface 202, to a user of the apparatus. Examples of the user interface comprise one or more or a combination of a speaker, a display, a touch screen, light source and a printer. The information output by the user interface may comprise one or more from an alarm, an electrocardiogram, a beat rate, HRV, an exercise level and a condition of the subject. The alarm may be a visual alarm or an audio alarm or a haptic alarm or a combination thereof. Examples of audio alarms comprise sounds, preferably with an audible volume level, e.g. at least 50 dB. Examples of visual alarms comprise graphical user interface elements for example symbols and light sources whose color may be set, e.g. red, to indicate an alarm. In an example, the haptic alarm may be caused by the apparatus 200 comprising an electric motor whose rotor is caused to generate vibration energy, e.g. by coupling the rotor with an eccentric element, to a housing of the apparatus. When the apparatus is worn by a user the vibration can be sensed by the user, whereby an alarm or other feedback may be communicated to the user. In an example, the alarm may be caused based on the estimated condition of the subject. In an example the output may comprise the communication interface 208 communicating output data, e.g. an ECG signal, or a beat rate signal and/or a measured IBI sequence to a remote apparatus. Additionally, the output data may comprise commands for controlling processing of the output data. Examples of the commands comprise at least one of the following: information indicating a level of match between first distributions and second distributions for estimating the condition of the subject; or at least one quantity characterizing the measured IBI sequence. In an example, the level of match may be associated with a condition of the subject, e.g. a healthy heart of the subject, a cardiac disease of the subject, a cardiac malfunction of the subject, an exercise load of the heart of the subject, drug exposure of the subject, sleep phase of the subject, stress level of the subject, and/or state of a nervous system of the subject. Accordingly, each condition of the subject may be determined based on comparing the first distributions and the second distributions, provided the associated level of match between has been met. If the level of match associated with the condition has not been met, estimation of the condition is not reliable, which may be output by the apparatus 200. In an example, the at least one quantity characterizing the measured IBI sequence may be at least one of the following: time instant or a time range for obtaining a time-dependent fluctuation function; or a physiological quantity derivable from the IBI sequence; or a physiological quantity measured from the subject concurrently with the IBI sequence. Accordingly, the at least one quantity characterizing the measured IBI sequence provides controlling processing of the output data, e.g. determining the first distributions and the second distributions.

The remote apparatus, e.g. a server, may host a computer program for processing the output data from the apparatus 200. In an example, the remote apparatus may process the measured IBI sequence received from the apparatus 200, use any commands received from the apparatus 200 for controlling the processing and send results of the processing back to the apparatus 200. The results may comprise e.g. an estimated condition of the subject. Using a separate remote apparatus for processing the output data enables off-loading processing from the apparatus 200 to the remote apparatus. In this way the condition of the subject may be estimated even if capabilities and/or processing resources of the apparatus 200 are limited. The remote apparatus may have sufficient resources for processing output data received from a plurality of apparatuses. An example of the remote apparatus is a cloud computing service.

It should be appreciated that the apparatus 200, for example an electrocardiogram monitoring device or a wearable monitoring device, may be further caused to display, by the user interface, one or more results determined based on an electrocardiography recording performed by the device or based on a beat rate measurement performed by the device, together with the estimated condition of the subject. In this way, the user of the apparatus may be assisted to correctly evaluate results of the electrocardiography recording or the beat rate measurement, and any alarm caused by the apparatus e.g. based on the estimated condition of the subject for continued interaction with the apparatus. The results of the electrocardiography recording or the beat rate measurement may be displayed by the user interface e.g. together with the estimated condition of the subject. Examples of the results of the electrocardiography recording comprise or at least indicate: an electrocardiogram, sinus rhythm, sinus tachycardia, sinus bradycardia, atrial fibrillation, atrial flutter, ventricular, tachycardia, ventricular fibrillation and/or heart rate. Examples of the results of the beat rate measurement comprise or at least indicate: a beat rate, a HRV and an exercise level. In an example, when the estimated condition of the subject is displayed with the results of the electrocardiography recording, the user may be assisted to correctly interpret the results of the electrocardiography recording. In an example, when the estimated condition of the subject is displayed with the results of the beat rate measurement, the user may be assisted to correctly interpret the results of the beat rate measurement. In another example, displaying the estimated condition of the subject may provide that the user may be assisted to interpret an alarm caused by the device and to determine to input a command to reset or not to reset the alarm such that continued use of the apparatus may be facilitated.

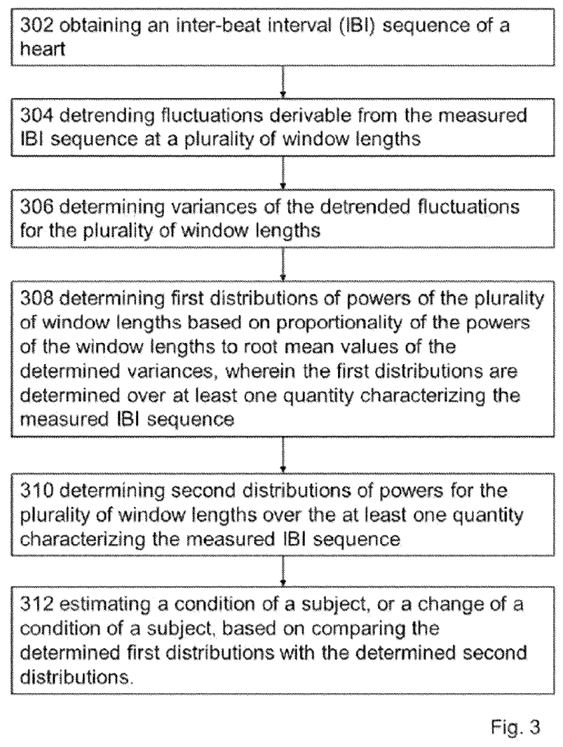

FIG. 3 shows, by way of an example, a flowchart of a method 300 for estimating condition of a subject. The method according to the present invention comprises a step 302 of obtaining an inter-beat interval (IBI) sequence of a heart. In a preferred embodiment the IBI sequence may be obtained by any suitable means or apparatus capable of measuring a beat rate, whereby the IBI sequence may be determined based on a beat rate signal measured e.g. based on electrical activity of the heart or based on photoplethysmography. In an embodiment, the IBI sequence may be obtained by measuring, e.g. by a wearable monitoring device, a heart monitoring device or an electrocardiogram monitoring device, a signal representing electrical activity of the heart of a subject and determining an IBI sequence from the signal. Examples of the signal comprise at least an ECG signal and a beat rate signal. In an embodiment, the IBI sequence, or information for determining the IBI sequence such as an ECG signal or a beat rate signal, may be received from a transmitting device over a data transfer connection. The data transfer connection may be a data network connection, for example an Internet Protocol (IP) connection. The transmitting device may be a wearable monitoring device, a heart monitoring device or an electrocardiogram monitoring device, or a computer connected to a wearable monitoring device, a heart monitoring device or an electrocardiogram monitoring device for receiving the IBI sequence.

The method further comprises a step 304 of detrending fluctuations derivable from the measured IBI sequence at a plurality of window lengths. In an example, the fluctuations may be derived based on comparing a time series with a trend within windows of the time series at the plurality of window lengths. The time series may be the IBI sequence or a time series derived based on the IBI sequence, for example an integrated time series obtained from the IBI sequence based on cumulative summation.

The method further comprises a step 306 of determining variances of the detrended fluctuations for the plurality of window lengths.

The method further comprises a step 308 of determining first distributions of powers of the plurality of window lengths based on proportionality of the powers of the window lengths to root mean values of the determined variances, wherein the first distributions are determined over at least one quantity characterizing the measured IBI sequence.

The method further comprises a step 310 determining second distributions of powers for the plurality of window lengths over the at least one quantity characterizing the measured IBI sequence. In this way the second distributions are determined for the same window lengths than the first distributions.

An example implementation of steps 304, 306, 308 and 310 is described by a detrended fluctuation analysis (DFA).

### DFA

DFA provides that collective behaviour of the obtained IBI sequence may be characterized by a single value, a scaling exponent α. In particular, DFA considers collective correlations within windows of specific number of steps. This is in contrast to the correlations determined by, e.g., the autocorrelation function that considers pointwise correlations that are specific number of steps (“lag”) apart from each other.

The DFA processes the obtained IBI sequence that is a time series X of N samples, i.e., X=(x₁, x₂, . . . , xₙ). The IBI sequence is uniformly sampled in the sense that the intervals correspond to subsequent beats one after another, i.e., the unit of “time” in this time series is a single beat. First, a cumulative summation is performed:

$${y}_{i} = {\sum }_{j = 1}^{i} ( {x}_{j} - 〈 X 〉 ) , \qquad ( 1 )$$

where xⱼ is an individual interbeat interval (IBI), <X> is an arithmetic mean of the original time series, and Nis a number of IBI samples in a processed segment of the IBI sequence. In this way an integrated time series Y=(y₁, y₂, . . . , y_{N}) is obtained. When each IBI sample is interpreted as a step length of a random walk theory, the time series Y represents a random walk. This facilitates interpretation of scaling exponent for estimating a condition of a subject. Formula (1) is an example, of the IBI sequence obtained in phase 302 of FIG. 3.

Detrended variances of the fluctuations of the IBI sequence from a local trend may be calculated. A trend may be determined by a least-squares fit of a low-order polynomial to data. The detrended variances of fluctuations are computed as the variance from the trends within each window:

$${F}_{s , i}^{2} = \frac{1}{s} {\sum }_{j = i}^{i + s} {[ {y}_{j} - {f}_{s , i} ( j ) ]}^{2} \qquad ( 2 )$$

where [.] gives the fluctuation for a local trend within a window i for index j, s is the window length, or scale, and i is index of the window. Scale s is the length of the window, in number of consecutive elements of the time series, through which the behaviour of the time series is studied. In the random walk formalism scale s is equivalent to the number of steps taken. f_{s,i} (j) is the trend within the window at scale s and index i. The indices i may take values i∈{1, 2, . . . , N−s+1}. In this notation, index i corresponds to the index of the first element yᵢ of the time series Y that belongs to a particular window of length s. Furthermore, conventionally the windowing is non-overlapping, i.e., only indices i=1, s+1, 2s+1, 3s+1, . . . would be included. However, the statistical properties of the analysis may be improved by maximally overlapping windows, i.e., considering all the possible windows (in the IBI sequence), where i={1, 2, . . . , N-s+1}, at increased computational cost. Formula (2) is an example, of processing the obtained IBI sequence according to phases 304 and 306 of FIG. 3.

The detrended variances can be used to calculate a fluctuation function F(s):

$$F ( s ) = \sqrt{〈 {F}_{s , i}^{2} 〉} , \qquad ( 3 )$$

where the angle brackets denote the arithmetic mean (computed over all the indices i separately for each scale s).

Instead of considering behaviour over the complete time series, it is often beneficial to study how the fluctuations behave locally as a function of a quantity of interest q. To facilitate this dynamic study of the fluctuation function F(s,q) as the function of the quantity of interest, we introduce a dynamic segmentation function S(s,q) that returns the set of indices i for the detrended variances F_{s,i}² that correspond to segments of the time series where the quantity of interest has the value q:

$$F ( s , q ) = \sqrt{〈 {F}_{s , i}^{2} 〉} , i ∈ S ( s , q ) , \qquad ( 4 )$$

where mean of the detrended variances is calculated over the indices i in the set S(s,q). It should be noted that since the dynamic segmentation function is dependent on q, i.e. a quantity characterizing the obtained IBI sequence, the segmentation of the detrended variances may be adapted based on q. For practical implementations it may be necessary to consider a range of values within the neighbourhood of the value q to obtain sufficient statistical significance for the estimate of the local fluctuation function.

Allowing the windows to overlap enhances the statistical properties of this estimate, i.e., F(s). The procedure may be repeated for different window sizes, or scales s. The fluctuation function F(s) may follow the power-law, whereby F(s)˜s^{a}, where α is the scaling exponent.

It is a known result from the random walk theory that for an uncorrelated walker we get F(s)∝

$${s}^{\frac{1}{2}} .$$

A more general case with correlated (or anticorrelated) steps may be characterized by a scaling exponent

$$α \neq \frac{1}{2} :$$

$$F ( s ) ∝ {s}^{α} \qquad ( 5 )$$

Correlated steps, i.e., the steps are more likely to be to the same direction with approximately the same magnitude, result in

$$α > \frac{1}{2}$$

with higher values indicating higher degree of correlations. Consequently, anticorrelated steps, i.e., the steps are more likely to be to opposite directions with approximately the same magnitudes, result in

$$α < \frac{1}{2}$$

with lower values for greater anticorrelations.

In conventional DFA, the power law scaling of Eq. 5 is transformed into linear relationship by a logarithmic transformation. The scaling exponent α is determined as the slope of a simple regression line fit on a log-log plot of the fluctuation function versus scale. In the context of HRV, two scaling exponents are conventionally determined: Short-scale α₁ for scales 4-16 and long-scale α₂ for scales 16-64.

Many processes, including HRV, do not exhibit strict scaling over all the scales. Therefore, instead of relying on line-fitting over ranges of scales, it is useful to study a whole spectrum of exponents α(s) as the function of the scale s. The scale-dependent exponent is conveniently defined as the local slope of the logarithmic fluctuation function on logarithmic scale

$$α ( s ) = \frac{d [ log F ( s ) ]}{d [ log s ]} , \qquad ( 6 )$$

where the derivatives are first calculated with respect to the logarithmic scale and then evaluated at scale s. The formula can be expanded for a dynamic case with α(s, q) computed utilizing the dynamic fluctuation functions F(s, q).
The first and second distributions of powers are determined as distributions of the spectrum of scaling exponents α(s, q) as the function of the scale s and quantity of interest q. The resulting distributions of scaling exponents α are typically represented by a suitable mean or average value of the scaling exponent α, such as a median value or an arithmetic mean value.

In an embodiment, phase 308 comprises that the at least one quantity characterizing the obtained IBI sequence is at least one of the following: time instant or a time range for obtaining time-dependent fluctuation function, e.g. F(s, t) at time tor in the neighbourhood of time point t; a physiological quantity derivable from the IBI sequence; or a physiological quantity measured from the subject concurrently with the IBI sequence. In an example, the physiological quantity derivable from the IBI sequence may be a beat rate that may be computed from the IBI time series without a need for additional measuring devices. In an example, the physiological quantity measured from the subject concurrently with the IBI sequence may be a breathing rate that is typically available from simple heart rate (HR) monitors and fitness trackers.

The method of the invention further comprises a step 312 of estimating a condition of a subject, or a change of a condition of a subject, based on comparing the determined first distributions with the determined second distributions. In this way cardiac health of the subject may be assessed. In an example, the change of the condition of the subject may be a progression of a cardiac disease, effect of drug exposure, a change of an exercise load on the heart, a change in an aerobic or an aerobic energy production during exercise, a change of a sleep phase of the subject. In an example, the estimated condition of the subject may be a condition that is indicated with a high likelihood based on a comparison of the first distributions and the second distributions. It should be noted that the first distributions and the second distributions may be obtained from a single IBI measurement that is used to determine both the first distributions and the second distributions. For example, the first distributions may be determined based on a first portion of IBI measurements used for determining the first distributions, and the first portion of the IBI measurements precede in time a second portion of the IBI measurements used for determining the second distributions. In an embodiment, the method comprises: determining a subset of the determined variances based on a dynamic segmentation function that is dependent on window length and the at least one quantity characterizing the obtained IBI sequence; and determining the first distributions of powers of the plurality of window lengths based on the determined subset. In this way, estimation of the condition of the subject may be controlled based on the window length and the at least one quantity of interest. In an embodiment, the subset is determined based on values of the at least one quantity characterizing the obtained IBI sequence. For example, if the condition of the subject is known to take place during exercise, the subset may be determined using heart rate as the quantity of interest and particularly heart rate values that take place during exercise. In an embodiment, the subset of determined variances is a proper subset of a set of determined variances, i.e., the subset is unequal to the set of determined variances. In an embodiment, the second distributions are reference distributions determined based on one or more IBI sequences measured from one or more reference subjects having a clinically validated condition and the first distributions are determined based on the measured IBI sequences, or the second distributions are reference distributions determined based on one or more IBI sequences measured from the same subject. In an example, the second distributions may be stored in a library that is accessible to an apparatus performing the method over a data network connection or the second distributions may be stored locally at the apparatus. At the apparatus, the second distributions may be stored locally to a data storage to the apparatus via a data bus. The library may be a computer-implemented data storage, e.g. provided by a cloud computing service, where second distributions corresponding to a plurality of clinically validated conditions are stored. Storing the second distributions in the library provides that the second distributions may be managed centrally. Alternatively, or additionally, a storage capacity of a data storage storing the library may be larger than that of the apparatus, whereby an amount of second distributions stored at the library may be larger in number or in size or both in number and size, than an amount of second distributions stored at the apparatus locally. For example, second distributions stored locally at the apparatus may correspond with a set of clinically validated conditions that is smaller than a set of clinically validated conditions that correspond to second distributions stored in a library. The apparatus may be connected to the library, whereby the apparatus may download/receive second distributions from the library. Accordingly, at the apparatus it may be determined one or more clinically conditions that are to be detected and the second distributions corresponding to the determined one or more clinical conditions may be retrieved from the library by the apparatus. If second distributions are not needed at the apparatus, they may be deleted and retrieved later from the library, when needed. In this way the second distributions stored locally may be optimized according to need. The first distributions may be based on obtained IBI sequences from a subject. Comparing the determined first distributions with the second distributions may reveal a condition of the subject, for example a condition of the heart of the subject. In an example, the second distributions are reference distributions determined based on one or more IBI sequences measured from the same subject may be without clinically validated condition but associated with information indicating one or more measurement conditions of the IBI sequence. Examples of the measurement conditions comprise at least the following: time, date, a beat rate, a HRV of the subject and an exercise level of the subject.

In an embodiment, phase 312 comprises: determining output data based on the result of the comparison of the determined first distributions with the determined second distributions; and providing the output data to a receiver entity for causing at least one operation of the receiver entity based on the determined output data. The output data may be transmitted over a data transfer connection, for example an Internet Protocol (IP) connection, or a Bluetooth connection. Alternatively or additionally, the output data may be transmitted on a data bus inside an apparatus. The output data may be used at the apparatus or at the receiver entity to cause output of information to a user. Accordingly, e.g. a user interface may be controlled based on the output data. The output of information may comprise displaying the estimated condition of the subject. The estimated condition of the subject may be displayed together with at least one of the following: results of an electrocardiography recording; or results of a beat rate measurement; or output of an alarm. Examples of the receiver entity comprise at least a processor, a user interface, cloud computing service and a smart phone.

In an embodiment, the condition of the subject comprises a healthy heart of the subject, a cardiac disease of the subject, a cardiac malfunction of the subject, an exercise load of the heart of the subject, drug exposure of the subject, sleep phase of the subject, stress level of the subject, and/or state of a nervous system of the subject.

In an embodiment, each clinically validated condition of the reference subject may be selected from a healthy heart, a cardiac disease, a cardiac malfunction, an exercise load of the heart, drug exposure, sleep phase, stress level, state of a nervous system, or any combination thereof.

In an embodiment, the method 300, e.g. at step 312, comprises:
determining at least one characteristic region for the determined second distributions;
comparing the determined first distributions with the determined at least one characteristic region; and
estimating the condition of the subject based on a result of the comparison between the determined first distribution with the determined at least one characteristic region. In an example, the at least one characteristic region may have been determined based on comparing positive distributions and negative distributions obtained from subjects based on IBI sequences as described in phases 308 to 310. The positive distributions may have been determined based on IBI sequences from subjects that have a specific condition. The negative distributions may have been determined based on IBI sequences from subjects that do not have the specific condition. Whether a subject has or does not have the condition may have been verified e.g. by clinical studies. Examples of the conditions comprise at least one of the following: a healthy heart of the subject, a cardiac disease of the subject, a cardiac malfunction of the subject, an exercise load of the heart of the subject, drug exposure of the subject, sleep phase of the subject, stress level of the subject, and/or state of a nervous system of the subject.

In an embodiment, the method 300, e.g. at step 312, comprises:
determining the condition of the subject based on the determined first distributions comprising the determined at least one characteristic region.

In an example, in phase 312, the at least one characteristic region for the determined second distributions may be typical of a certain clinically validated condition of the subject. Accordingly, the at least one characteristic region is typically present in the determined second distributions, when a subject has certain clinically validated condition. The comparison may provide information that indicates a presence of the at least one characteristic region, or a region corresponding to the at least one characteristic region, in the first distributions. It should be noted that a clinically validated condition may be associated with one or more characteristic regions within a determined second distribution. In other words, each characteristic region within a determined second distribution may be associated with a clinically validated condition of a subject. Preferably, the one or more characteristic regions associated with different clinically validated conditions differ from each other as to distinguish between the different clinically validated conditions. Each clinically validated condition may be associated with a unique, fingerprint-like characteristic region or a collection of characteristic regions for a determined second distribution. In an example, comparing the determined first distributions with the determined at least one characteristic region comprises comparing one or more characteristic regions of the determined first distributions with one or more characteristic regions of the determined second distribution.

Comparing the determined first distributions with the determined at least one characteristic region enables to identify whether the characteristics of the determined first distributions based on IBI sequence obtained from the subject are similar to the at least one characteristic region of the second distribution. Estimating the condition of the subject relies on a result of the comparison. Similarities in the characteristics of the determined first distributions when compared to the at least one characteristic region are an indication that condition of the subject is similar to the clinically validated condition of the reference subject.

It should be noted that, the characteristic region may be at least one of the following: a high density region of a distribution, or a low density region of a distribution. A collection of characteristic regions may be formed by one or more high density regions, or one or more low density regions, or one or more high density regions and one or more low density regions. The characteristic region may be a uniform region at which a density of the distribution corresponds with a range of values. The density may be quantified based on a frequency of occurrence of scaling exponent values in a neighbourhood of certain scales and values of the at least one quantity of interest.

In an embodiment, phase 312 comprises that the second distributions comprise a plurality of characteristic regions and the condition of the subject is estimated based on a level of match between the first distributions and the plurality of characteristic regions. In an example, the level of the match may be calculated based on a distance metric. The distance metric may be defined in various ways. For example, if the distributions are digital images, the distance metric may be defined for image data and the level of match may be determined based on image processing techniques, e.g. image recognition. In an example, an image formed based on the first distributions may be subtracted from an image formed based on the second distributions. If a resulting image comprises uniform areas that each have a smaller size than sizes of the characteristic regions of the second distributions, the level of match may be determined to be sufficient for estimating the condition of the subject to correspond with a clinically validated condition corresponding to the second distributions. It should be noted that the first distributions and the second distributions may be extracted from the same image. Accordingly, a first portion of the image may comprise the first distributions and a second portion of the image may comprise the second distributions.

In an embodiment, phase 312 comprises comparing the determined first distributions to several different second distributions associated with different clinically validated conditions stored in a library, or comparing the determined first distributions to second distributions obtained from the same subject. Comparing the determined first distributions and each of the several different second distributions facilitates searching for similarities between the determined first distributions and the several different second distributions. Therefore, a condition of the subject may be indicated by second distributions that have been determined based comparison of the first distributions and each of the several different second distributions to be most similar to the determined first distribution. Then, the condition of the subject may be determined to be a clinically validated condition corresponding to the second distributions that are most similar to the determined first distributions. Comparing the determined first distributions to second distributions obtained from the same subject enables self-assessment of the subject. In this case, the second distributions may be without a clinically validated condition but associated with information indicating one or more measurement conditions of the IBI sequence. Examples of the measurement conditions comprise at least the following: time, date, a beat rate, a HRV of the subject and an exercise level of the subject. In this way the first distributions and the second distributions may be used for self-assessment of the condition of the subject without distributions of clinically validated conditions requiring measurements from other subjects.

The invention further provides an apparatus. The apparatus comprises:
means for obtaining an inter-beat interval (IBI) sequence of a heart;
detrending fluctuations derivable from the obtained IBI sequence at a plurality of window lengths;
means for determining variances of the detrended fluctuations for the plurality of window lengths;
means for determining first distributions of powers of the plurality of window lengths based on proportionality of the powers of the window lengths to root mean values of the determined variances, wherein the first distributions are determined over at least one quantity characterizing the obtained IBI sequence;
means for determining second distributions of powers for the plurality of window lengths over the at least one quantity characterizing the obtained IBI sequence; and
means for estimating a condition of a subject, or a change of a condition of a subject, based on comparing the determined first distributions with the determined second distributions.

In an embodiment, the means of the apparatus are configured to perform the method according to the invention.

In an embodiment, the means comprise at least one processor and at least one memory including computer program code. In an embodiment, the at least one memory and the computer program code are configured to, with the at least one processor, cause the performance of the apparatus. In an embodiment, the at least one memory and the computer program code are configured to, with the at least one processor, cause the apparatus to perform the method according to the invention.

In an embodiment, the apparatus is a server, a smart phone, a wearable monitoring device, a heart monitoring device or an electrocardiogram monitoring device. In an embodiment, the wearable monitoring device may be worn, e.g., around a torso or a limb, such as a wrist, an upper arm, or a leg of the subject.

A computer program according to the invention comprises computer program code that is configured to, when executed on at least one processor, cause an apparatus to perform the method of the invention.

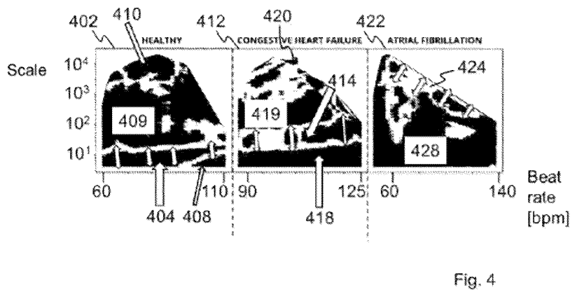

FIG. 4 shows, by way of example, distributions 402, 412, 422 that have been determined based on obtained IBI sequences. It should be noted that although the distributions are shown by images, processing of the distributions may be carried out by numerical methods without image data. The images are shown by 2D (two-dimensional) images, where each point, or pixel, in the images represents a distribution of scaling exponent values. For example, each point may be an average value calculated from a distribution of scaling exponent values associated with a given point. Therefore, each point may be examined at a level of the average value calculated from the distribution associated with the point or at a level of the associated distribution. In an example, the distributions may be determined in accordance to described with steps 302, 304, 306 and 308. Each of the distributions comprise at least one characteristic region. The distributions may be compared with each other based on their characteristic regions for estimating a condition of the subject. For example, if a comparison between distributions based on their characteristic regions indicates a match between the distributions, the distributions may be determined to match with the same condition of the subject. In an example, if one distribution, i.e. a distribution under study, that has been determined based on an obtained IBI sequence, is compared with another distribution that corresponds with a clinically validated condition, the distribution under study may be determined to indicate the clinically validated condition based on a match between characteristic regions of the distributions. The distribution under study may be obtained from IBI measurements from a subject, whereby a condition of the subject may be estimated to be the clinically validated condition.

The characteristics regions may extend over an area defined by a range of values of scales and a range of values of a quantity characterizing an obtained IBI sequence. In the illustrated example, the quantity characterizing an obtained IBI sequence is beat rate [bpm]. Therefore, the distributions show powers of window lengths corresponding to values of window length and beat rate [bpm].

The characteristic region may be a uniform region at which values of the distribution corresponds with a range of values. In an example, the characteristic region may be at least one of the following: a high value region of a distribution, or a low value region of a distribution. A collection of characteristic regions may be formed by one or more high value regions, or one or more low value regions, or one or more high value regions and one or more low value regions.

The distributions comprise a distribution 402 corresponding to a healthy heart of the subject, a distribution 412 corresponding to a congestive heart failure and a distribution 422 corresponding to an atrial fibrillation.

The distribution corresponding to the healthy heart comprises a characteristic region 404. The characteristic region 404 may be a region, where values of the distribution are higher than at a region 408 below the characteristic region 404 or a region 409 above the characteristic region. The characteristic region 404 may extend from 60 bpm to 110 bpm and scales 0 to 11. The characteristic region 404 is illustrated by double-headed arrows. Accordingly, the regions 408 and 409 are low value regions. The low value region 408 extends from 95 bpm to at least 110 bpm and in scales from 0 to 8 or 9. The low value region 409 extends from 60 bpm to at least 100 bpm, or from 60 bpm to at least 110 bpm, and in scales from 11-13 to about 1000. Region 410 that has the highest values of scales, is separated from the characteristic region 404 by the low value region 409. The region 410 extends from 65 bpm to at least 90 bpm and in scales from 1000 to at least 10000.

The characteristic region 414 may be a region, where values of the distribution are higher than at a region 418 below the characteristic region 414 or a region 419 above the characteristic region. Accordingly, the regions 418 and 419 are low value regions. As a difference to the distribution of the healthy heart 402, the characteristic region 414 of the congestive heart failure extends over an area defined by beat rate, e.g. from 90 bpm to 115 bpm, more particularly from 85 bpm to 115 bpm, more particularly from 85 bpm to 125 bpm. Regarding the scales the characteristic region 414 extends over an area defined by scales, e.g. from 10 to about 100. At highest values of the scales, above the low value region 419, a uniform area of region 420 is smaller than with the distribution of the healthy heart 402. The low value region 419 extends from 85 bpm to at least 110 bpm and in scales from 600 to at least 10000. The low value region 418 extends from 85 bpm to 125 bpm and in scales from 0 to 10 or 11.

The characteristic region 424 may be a region, where a value of the distribution is higher than at a region 428 below, e.g. at lower values of the scales, the characteristic region 424. Accordingly, the region 428 is a low value region. As a difference to the distribution of the healthy heart 402, the low value region extends over an area defined by beat rate, e.g. from 40 bpm to 140 bpm and in scales from 0 to 100, and at a portion of the beat rates up to scale 1000.

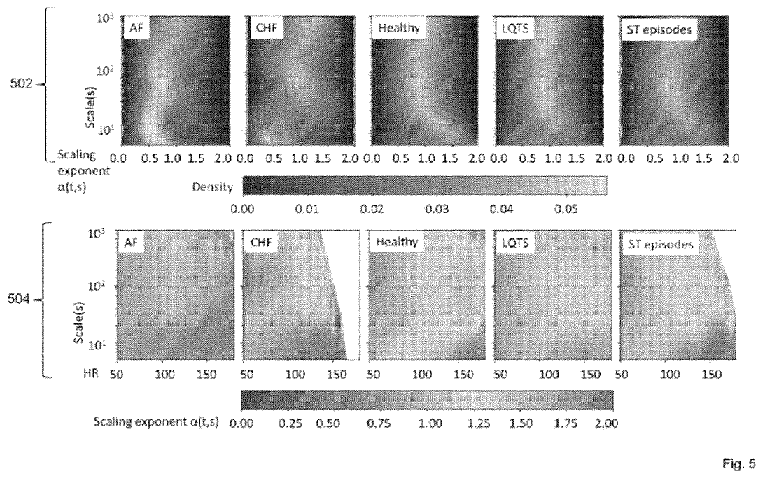

Examples of applying a method in accordance with at least some embodiments for estimating a condition of the subject is provided in FIGS. 5, 6, 7 and 8. FIGS. 5, 6, 7 and 8 illustrate distributions that have determined in accordance to described with steps 302, 304, 306 and 308 implemented by a detrended fluctuation analysis (DFA). Further examples of application of the DFA for estimating a condition of the subject regarding physiological exercise thresholds can be referred to in Kanniainen et al. 2023a and for estimating a condition of the subject regarding sleep phase and state of a nervous system Kanniainen et al. 2023b.

FIG. 5 shows examples of aggregated distributions of healthy subjects and subjects with Long QT syndrome (LQTS), Atrial Fibrillation (AF), Congestive Heart Failure (CHF), ST episodes with distributions 502 as function of the scaling exponents α(t,s) and with distributions 504 as function of the heart rate (HR).

The aggregated distribution as function of the scaling exponents α(t,s) from healthy subjects has a high density region at high scales with scaling exponent values concentrated around 1.0. The aggregated distributions from subjects with AF has a high density region at lower values of scaling exponent than the aggregated distributions from healthy subjects 502. The aggregated distributions from subjects with CHF, LOTS and ST episodes have high density regions at broader values of scaling exponent than the aggregated distributions from healthy subjects.

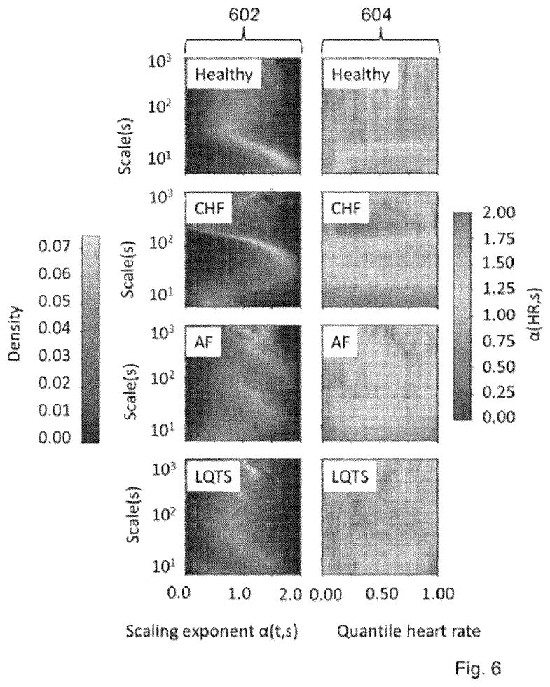

The aggregated distribution 504 as function of the heart rate from healthy subjects has a region of high scaling exponent α(t,s) values covering a broad range of scales with HR above 100. The aggregated distributions from subjects with AF are almost completely missing a region of high scaling exponent α(t,s) values. The aggregated distributions from subjects with LOTS has somewhat higher scaling exponent α(t,s) values than the aggregated distributions from subjects with AF, but scaling exponent α(t,s) values are typically lower when compared with the aggregated distributions as function of the heart rate from healthy subjects. Compared with the the aggregated distributions as function of the heart rate from healthy subjects, the aggregated distributions from subjects with CHF and ST episodes have less high scaling exponent α(t,s) values at low scales, e.g. s<100. FIG. 6 shows examples of distributions for a healthy subject and single subjects with Long QT syndrome (LQTS), Atrial Fibrillation (AF), Congestive Heart Failure (CHF), with distributions 602 as function of the scaling exponents α(t,s) and with distributions 604 as function of the quantile heart rate.

The distribution as function of the scaling exponents α(t,s) from healthy subject has a narrow high density region at low scales and a broader high density region at higher scales, e.g. s>300. The distributions from subjects with AF and LOTS have more scattered high density regions at scales s<300 than the distribution from the healthy subject. The distributions from subject with CHF has scattered high density regions at low scales and high scales and between the high scales and low scales the scattering is reduced.

The distribution as function of the quantile heart rate from healthy subject has a region of high α-values, e.g. α>1.50, at scales s<200 and almost over the whole range of the quantile hear rate. At small heart rates, e.g. at resting hear rate, α<1.0, e.g. around 0.5. The distributions from subjects with CHF and LQTS have scattered high density region over the scales and the quantile heart rate. Furthermore, the distributions from subjects with CHF and LOTS have less high values of α, e.g. α>1.75, than the distribution as function of the quantile heart rate from healthy subject. The distribution from subjects with CHF have a region of high α values clearly distinguished between regions of lower a values above and below the region of high α values.

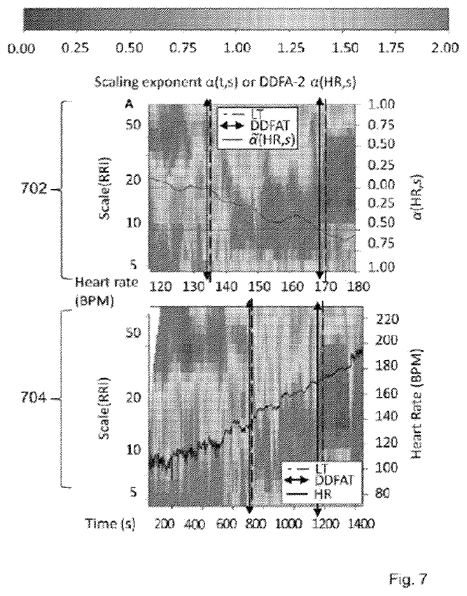

FIG. 7 shows examples of distributions for determining exercise load of a subject. Details of determining the exercise load according to FIG. 7 can be referred to in Kanniainen et al. 2023a. The distributions provide examples of application of the DFA for estimating an exercise load of the heart of the subject, a change of an exercise load on the heart and a change in an aerobic or an aerobic energy production during exercise. The distributions are determined from the subject based on data obtained from an exercise test. The distributions 702, 704 show aggregated results from the subject as function of the heart rate and as function of time. In FIG. 7A the distribution 702 shows the scaling exponent as functions of HR (x-axis) and scale (y-axis), whereas in FIG. 7B the distribution 704 shows the scaling exponent as functions of time and scale. In FIG. 7A the scaling exponent values, or α-values, are high at heart rate 120 BPM and with scales below 30. At heart rate at about 135 BPM, the α-values are turned lower which can be observed as reduction of high α-values of 1.75 and above at heart rates above ˜135 BPM compared with heart rates below ˜135 BPM. At heart rate above 170 BPM, at scales from 10 to 40 α-values less than 0.25 are clearly increased compared with α-values at lower heart rates.

In FIG. 7B the scaling exponent values, or α-values, are high about up to time 600 and at scales less than 20. After time 600 the high α-values start to reduce and after about time 800, the preceding region of high α values up to time 600 is missing. At time values following 1200, a region on scales from 10 to about 30 is presented with α-values less than 0.25. Thus α-values less than 0.25 have increased compared with the α-values from 800 to 1200.

The solid line in FIG. 7A shows the mean a (HR) used in the determination of exercise thresholds, referred to dynamic detrended fluctuation analysis (DDFA) thresholds. FIG. 7A shows two DDFA thresholds DDFAT1 and DDFAT2 that are illustrated by arrow-headed lines together with benchmark lactate concentration (LT) values shown in black vertical dashed lines. In this particular example DDFAT thresholds underestimate LT1 and LT2 by only one and three BPM, respectively. The DDFAT1 is the point, where the scaling exponent distribution, α⁻(HR) distribution, drops below a baseline. The baseline corresponds to α(HR,s) in resting conditions of the subject. The determination of this point can be derived through the following calculation. First find the points where α⁻(HR) distribution crosses a baseline. Then, if the intersection is not stable, i.e., α⁻(HR) keeps fluctuating around the baseline, move to the next one until a stable intersection is found. Sufficient stability can be found when α⁻(HR) remains negative for at least 10 consecutive HR values. The DDFAT2 is determined at a stable intersection of the α⁻(HR) and the baseline, where α⁻(HR) equals −0.5. The baseline can be determined from the mean value of α(HR,s) with the lowest HR for each scale s. The baseline values of each scale are subtracted from α(HR,s) over the whole measurement, whereby α⁻(HR) is obtained.

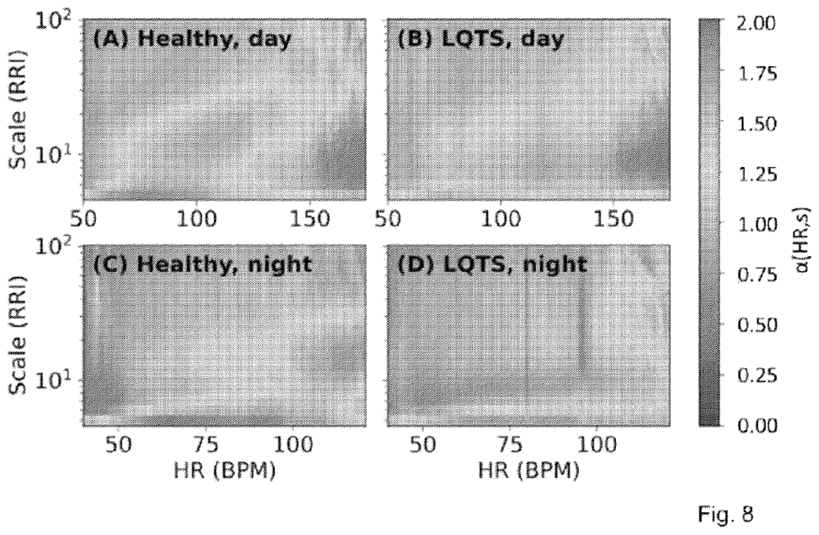

FIG. 8 shows examples of aggregated day and night distributions for healthy subjects and subjects with LOTS. Details of aggregated day and night distributions for healthy subjects and subjects with LOTS can be referred to in Kanniainen et al. 2023b. The distributions provide examples of application of the DFA for estimation of state of a nervous system of the subject, sleep phase of the subject and state of a nervous system of the subject their changes. The most distinguishable difference between the distributions is again visible during the day, where α(HR, s) is considerably higher for healthy than for LQTS subjects across a large range of HR (70-120 BPM), especially for the short scales (4-16), but also extending to scales up to 30. Moreover, during the night, the healthy subjects exhibit higher α (HR, s), especially for higher HR values. However, the short-scale differences are reduced, potentially leading to reduced discrimination power. It is also noteworthy that the maximal HRs found at night are considerably lower than during the day, and thus the different axes between the day- and night-time are not directly compatible.

### CHF

Congestive heart failure (CHF), often referred to as heart failure, is a chronic condition in which the heart is unable to pump blood efficiently to meet the body's needs. This results in a reduced ability of the heart to fill with or eject blood, leading to a cascade of symptoms and complications.

### AF

Atrial fibrillation (AF) is a common heart rhythm disorder characterized by irregular and often rapid heartbeats. In atrial fibrillation, the heart's upper chambers (atria) experience chaotic electrical signals, leading to an irregular and sometimes rapid heartbeat. The irregular heart rhythm can cause poor blood flow to the body, increasing the risk of stroke and other heart-related complications. Atrial fibrillation can be categorized into different types based on its duration and whether it occurs spontaneously or is triggered. Types include paroxysmal, persistent, long-standing persistent, and permanent atrial fibrillation.

### LQTS

Long QT syndrome (LQTS) is a cardiac disorder characterized by an abnormality in the heart's electrical system, specifically the time it takes for the ventricles to repolarize after a heartbeat. The term “QT” refers to the interval on an electrocardiogram (ECG) between the start of the Q wave and the end of the T wave.

### ST Episode

The ST episode refers to changes observed in the ST segment on an electrocardiogram (ECG). The ST segment is a portion of the ECG waveform that represents the interval between ventricular depolarization and repolarization. ST-segment changes can be indicative of various cardiac conditions. Common scenarios associated with ST segment changes comprise:
ST-segment elevation (ST elevation): This is often a sign of myocardial infarction (heart attack). When the blood flow to a part of the heart muscle is blocked, the affected area can exhibit ST-segment elevation on the ECG.
ST-segment depression: This may occur in conditions such as myocardial ischemia (inadequate blood flow to the heart muscle) or angina pectoris.
Flat ST segment: A flat ST segment may be considered abnormal and could be associated with various cardiac and non-cardiac conditions.
ST-segment changes are crucial in diagnosing and monitoring heart conditions. If you or someone you know is experiencing ST-segment changes on an ECG, it's important to seek medical attention promptly for further evaluation and appropriate management.

Embodiments may be implemented in software, hardware, application logic or a combination of software, hardware and application logic. The software, application logic and/or hardware may reside on memory, or any computer media. In an example embodiment, the application logic, software or an instruction set is maintained on any one of various conventional computer-readable media. In the context of this document, a “memory” or “computer-readable medium” may be any media or means that can contain, store, communicate, propagate or transport the instructions for use by or in connection with an instruction execution system, apparatus, or device, such as a computer.

The foregoing description has provided by way of exemplary and non-limiting examples a full and informative description of the exemplary embodiment of this invention. However, various modifications and adaptations may become apparent to those skilled in the relevant arts in view of the foregoing description, when read in conjunction with the accompanying drawings and the appended claims. However, all such and similar modifications of the teachings of this invention will still fall within the scope of this invention.

### REFERENCES

Kanniainen et al. 2023a: Matias Kanniainen, Teemu Pukkila, Joonas Kuisma, Matti Molkkari, Kimmo Lajunen, and Esa Räsänen, Estimation of Physiological Exercise Thresholds Based on Dynamical Correlation Properties of Heart Rate Variability, Frontiers in Physiology, Vol. 14 (2023); https://doi.org/10.3389/fphys.2023.1299104.
Kanniainen et al. 2023b: Matias Kanniainen, Teemu Pukkila, Matti Molkkari, and Esa Räsänen, Effect of Diurnal Rhythm on RR Interval Correlations of Long QT Syndrome, Computing in Cardiology, Vol. 14 (2023); https://cinc.org/2023/Program/accepted/287_CinCFinalPDF.pdf.

1. A method comprising:
obtaining an inter-beat interval (IBI) sequence of a heart of a subject;
deriving a time series based on the IBI sequence;
introducing a dynamic segmentation function as a function of a quantity of interest, wherein the dynamic segmentation function returns a set of indices that correspond to segments of the time series;
detrending fluctuations derivable from the obtained IBI sequence at a plurality of window lengths as a function of the quantity of interest, whereby the window lengths are defined in number of consecutive elements of the time series by
determining variances of the detrended fluctuations for the plurality of window lengths at the indices returned by the dynamic segmentation function, wherein the indices correspond to segments of the time series;
calculating root mean values of the determined variances;
determining first distributions of powers for the plurality of window lengths based on proportionality of the powers of the window lengths to root mean values of the determined variances, wherein the first distributions are determined over at least one quantity of interest characterizing the obtained IBI sequence;
determining second distributions of powers for the plurality of window lengths over the at least one quantity of interest characterizing the obtained IBI sequence;
determining a plurality of characteristic regions for the determined second distributions;
comparing the determined first distributions with the determined plurality of characteristic regions; and
estimating a condition of the subject, wherein the condition of the subject comprises a healthy heart of the subject, a cardiac disease of the subject, a cardiac malfunction of the subject, an exercise load of the heart of the subject, drug exposure of the subject, sleep phase of the subject, stress level of the subject, and/or state of a nervous system of the subject, or a change of a condition of a subject, e.g., a progression of a cardiac disease, effect of drug exposure, a change of an exercise load on the heart, a change in an aerobic or an aerobic energy production during exercise, or a change of a sleep phase of the subject, based on a level of match between the determined first distribution and the plurality of characteristic regions.

2. The method of claim 1, comprising:
determining a subset of the determined variances based on the dynamic segmentation function that is dependent on window length and the at least one quantity characterizing the obtained IBI sequence; and
determining the first distributions of powers of the plurality of window lengths based on the determined subset.

3. The method of claim 2, wherein the subset is determined based on values of the at least one quantity characterizing the obtained IBI sequence.

4. The method of claim 1, comprising:
determining output data based on the result of the comparison of the determined first distributions with the determined second distributions; and
providing the output data to a receiver entity for causing at least one operation of the receiver entity based on the determined output data.

5. The method of claim 1, wherein the second distributions are reference distributions determined based on one or more IBI sequences measured from one or more reference subjects having a clinically validated condition and the first distributions are determined based on the obtained IBI sequence, or the second distributions are reference distributions determined based on one or more IBI sequences measured from the same subject.

6. The method of claim 1 comprising:
determining the condition of the subject based on the determined first distributions comprising the determined at least one characteristic region.

7. The method of claim 1, wherein the at least one quantity characterizing the measured IBI sequence is at least one of the following:
time instant or a time range for obtaining a time-dependent fluctuation function; or
a physiological quantity derivable from the IBI sequence; or
a physiological quantity measured from the subject concurrently with the IBI sequence.

8. The method of claim 1, comprising:
comparing the determined first distributions to several different second distributions associated with different clinically validated conditions stored in a library, or comparing the determined first distributions to second obtained from the same subject.

9. An apparatus comprising:
means for obtaining an inter-beat interval (IBI) sequence of a heart of a subject;
means for deriving a time series based on the IBI sequence;
means for introducing a dynamic segmentation function as a function of a quantity of interest, wherein the dynamic segmentation function returns a set of indices that correspond to segments of the time series;
means for detrending fluctuations derivable from the obtained IBI sequence at a plurality of window lengths as a function of the quantity of interest, whereby the window lengths are defined in number of consecutive elements of the time series;
means for determining variances of the detrended fluctuations for the plurality of window lengths at the indices returned by the dynamic segmentation function, wherein the indices correspond to segments of the time series;
means for calculating root mean values of the determined variances;
means for determining first distributions of powers of the plurality of window lengths based on proportionality of the powers of the window lengths to root mean values of the determined variances, wherein the first distributions are determined over at least one quantity of interest characterizing the obtained IBI sequence;
means for determining second distributions of powers for the plurality of window lengths over the at least one quantity of interest characterizing the obtained IBI sequence;
means for determining a plurality of characteristic regions for the determined second distributions;
means for comparing the determined first distributions with the determined plurality of characteristic regions; and
means for estimating a condition of the subject, wherein the condition of the subject comprises a healthy heart of the subject, a cardiac disease of the subject, a cardiac malfunction of the subject, an exercise load of the heart of the subject, drug exposure of the subject, sleep phase of the subject, stress level of the subject, and/or state of a nervous system of the subject, or a change of a condition of a subject, e.g., a progression of a cardiac disease, effect of drug exposure, a change of an exercise load on the heart, a change in an aerobic or an aerobic energy production during exercise, or a change of a sleep phase of the subject, based on a level of match between the determined first distribution and the plurality of characteristic regions.

10. (canceled)

11. The apparatus according to claim 9, wherein the means comprise at least one processor; at least one memory including computer program code; the at least one memory and the computer program code configured to, with the at least one processor, cause the performance of the apparatus.

12. The apparatus according to any of claim 9, wherein the apparatus is a server, a smart phone, a wearable monitoring device, a heart monitoring device or an electrocardiogram monitoring device.

13. (canceled)

14. A computer readable medium storing instructions that when executed by an apparatus cause:
obtaining an inter-beat interval (IBI) sequence of a heart of a subject;
deriving a time series based on the IBI sequence;
introducing a dynamic segmentation function as a function of a quantity of interest, wherein the dynamic segmentation function returns a set of indices that correspond to segments of the time series;
detrending fluctuations derivable from the obtained IBI sequence at a plurality of window lengths as a function of the quantity of interest, whereby the window lengths are defined in number of consecutive elements of the time series by
a. determining variances of the detrended fluctuations for the plurality of window lengths at the indices returned by the dynamic segmentation function, wherein the indices correspond to segments of the time series;
b. calculating root mean values of the determined variances;
determining first distributions of powers for the plurality of window lengths based on proportionality of the powers of the window lengths to root mean values of the determined variances, wherein the first distributions are determined over at least one quantity of interest characterizing the obtained IBI sequence;
determining second distributions of powers for the plurality of window lengths over the at least one quantity of interest characterizing the obtained IBI sequence;
determining a plurality of characteristic regions for the determined second distributions;
comparing the determined first distributions with the determined plurality of characteristic regions; and
estimating a condition of the subject, wherein the condition of the subject comprises a healthy heart of the subject, a cardiac disease of the subject, a cardiac malfunction of the subject, an exercise load of the heart of the subject, drug exposure of the subject, sleep phase of the subject, stress level of the subject, and/or state of a nervous system of the subject, or a change of a condition of a subject, e.g., a progression of a cardiac disease, effect of drug exposure, a change of an exercise load on the heart, a change in an aerobic or an aerobic energy production during exercise, or a change of a sleep phase of the subject, based on a level of match between the determined first distribution and the plurality of characteristic regions.
